SEQUENCE LISTING (8 sequences)

SEQ ID NO: 1 - DNA, 167 bp, Epstein-Barr virus
aggacctacgctgccctagaggttttgctagggaggagacgtgtgtggctgtagccacccgtcccgggtacaagtcccgggtggtgaggacggtgtctgtggttgtcttcccagactctgctttctgccgtcttcggtcaagtaccagctggtggtccgcatgtttt

SEQ ID NO: 2 - DNA, 167 bp, Epstein-Barr virus; identical to SEQ ID NO: 1

SEQ ID NO: 3 - DNA, 167 bp, Epstein-Barr virus; identical to SEQ ID NO: 1

SEQ ID NO: 4 - DNA, 167 bp, Epstein-Barr virus; identical to SEQ ID NO: 1

SEQ ID NO: 5 - DNA, 167 bp, Epstein-Barr virus; identical to SEQ ID NO: 1

SEQ ID NO: 6 - DNA, 167 bp, Epstein-Barr virus; identical to SEQ ID NO: 1

SEQ ID NO: 7 - DNA, 167 bp, Epstein-Barr virus; identical to SEQ ID NO: 1

SEQ ID NO: 8 - DNA, 15 bp, Artificial Sequence
  probe
ggcagcgtaggtcct

(19) United States
(12) Patent Application Publication
(10) Pub. No.: US 2011/0065094 A1
(43) Pub. Date: Mar. 17, 2011

(54) METHOD AND KIT FOR DETECTION/IDENTIFICATION OF VIRUS-INFECTED CELL

(75) Inventors: Hiroshi Kimura, Nagoya-shi (JP); Yukihiro Nishiyama, Nagoya-shi (JP)

(73) Assignee: National University Corporation Nagoya University, Nagoya-shi (JP)

(21) Appl. No.: 12/933,736

(22) PCT Filed: Mar. 17, 2009
(86) PCT No.: PCT/JP2009/001173

§ 371 Date: Dec. 6, 2010

(30) Foreign Application Priority Data
Mar. 21, 2008 (JP) 2008-073384

(51) Int. Cl. C12Q 1/70

(52) U.S. Cl. 435/5

(57) ABSTRACT

Disclosed is a means for specifically detecting and identifying a virus-infected cell in a floating cell system by a simple manipulation and with high sensitivity. A virus-infected cell can be detected and identified by the following steps (1) to (5): (1) adding a first labeled antibody which has been labeled with a first labeling substance and is directed against a cell surface antigen specific to a target cell to a sample and allowing the mixture to react; (2) immobilizing a protein in the presence of an RNAstabilizing agent; (3) treating the protein with a surfactant; (4) adding a labeled nucleic acid probe for a nucleic acid specific to a target virus to cause the hybridization; and (5) detecting a cell labeled with both the first labeled antibody and the labeled nucleic acid probe by flow cytometry.

### TECHNICAL FIELD

The present invention relates to a method for detecting and identifying a virus-infected cell, and to a kit for the method.

### BACKGROUND ART

Epstein-Barr virus (EBV) causes opportunistic lymphoma, malignant lymphoma, leukemia and the like. EBV expresses EBV-encoded small RNA1 (EBER-1) which is a virus-specific mRNA in a nucleus of an infected cell. For pathogenic tissues, detection of EBER-1 using an in situ hybridization (ISH) method and a method for identifying an EBV-infected cell by staining a surface antigen have been established. However, it is difficult to identify an infected cell in a blood/suspension cell system comprising small number of EBV-infected cell, and the diagnosis is rebuffed in many cases.

A peptide nucleic acid (PNA) has a structure in which a glycine backbone is covalently bound to a base, is more stable than DNA and RNA, and is able to hybridize to a nucleic acid. A technique for detecting an EBV-infected cell using flow cytometry (FCM) by using this PNA as a probe after fluorescent labeling has been reported (Non-patent Document 1). This PNA probe is commercially available as a kit for diagnosing pathological tissues and is now widely used in clinical practices (Epstein-Barr Virus (EBER) PNA Probe/Fluorescein, Code No. Y5200, Dako). The above-mentioned kit using an EBV-specific PNA probe was developed for use in a pathogenic tissue fixed on a slide, and is not suitable for a detection and a diagnosis using blood or a suspension cell as a sample. Furthermore, such use is not expected.

### Non-patent Document 1: T. Just et al., J Virol Methods 73 (1998) 163-174

### DISCLOSURE OF THE INVENTION

### Problem to be Solved by the Invention

The object of the invention is to provide a means for detecting and identifying a virus-infected cell specifically and with high sensitivity in a suspension cell system by convenient operations.

### Means for Solving the Problem

The inventors considered that a procedure comprising an ISH method and FCM (FCM/ISH method) in combination is effective as a means for detecting and identifying an EBV-infected cell in a suspension cell system, and did various studies. First, they considered that the reason why the procedure has not been practically used yet is due to the following problems. (1) Since an antibody reaction against a protein, a surface antigen, and hybridization to RNA, EBER-1 are carried out successively, a condition for fixing under which both the protein and RNA are stabilized is required. (2) It is difficult to set the condition of a reaction liquid so that the nucleic acid probe is stably hybridized to EBER-1 while the antigenicity of the surface protein is retained. (3) Since an existing nucleic acid probe (the component of the above-mentioned kit) uses FITC which emits weak fluorescence, the signal thereof is weak, and the detection is difficult even by FCM. Therefore, it is expected that detection is difficult when the probe is applied to a human clinical sample having a low amount of expression of EBER-1.

In order to solve these problems, various experiments were carried out. First, various fixing solutions for proteins were compared, and the optimal fixing condition was found. On the other hand, they have studied while focusing on the concentration of formamide, and found the optimal condition for hybridization. Furthermore, they have succeeded in improving detection sensitivity by amplifying a fluorescent intensity. Moreover, they have succeeded in multiple staining of EBER-1 and a cell surface antigen by applying FCM/ISH method to EBV-positive B cell strains, T cell strains, NK cell strains and clinical samples (peripheral blood mononuclear cell).

The present invention is mainly based on the above-mentioned achievement and finding, and is as follows.

[1] A method of detecting and identifying a virus-infected cell, comprising the following steps (1) to (5):

(1) a step of adding a first labeled antibody which has been labeled with a first labeling substance and is directed against a cell surface antigen specific to a target cell to a sample and allowing the mixture to react;

(2) a step of fixing a protein in the presence of an RNA-stabilizing agent;

(3) a step of treating the sample with a surfactant;

(4) a step of adding a labeled nucleic acid probe for a nucleic acid specific to a target virus to cause hybridization; and

(5) a step of detecting a cell labeled with both the first labeled antibody and the labeled nucleic acid probe by flow cytometry.

[2] The method according to [1], wherein the target virus is Epstein-Barr virus.

[3] The method according to [2], wherein the nucleic acid specific to the target virus is a small RNA encoded by Epstein-Barr virus (EBER).

[4] The method according to any one of [1] to [3], wherein the target cell is B cell, T cell or NK cell.

[5] The method according [4], wherein the sample is a blood sample.

[6] The method according to any one of [1] to [5], wherein the RNA-stabilizing agent is acetic acid.

[7] The method according to [6], wherein the step (2) is carried out under a condition in which the concentration of acetic acid is from 0.5% (v/v) to 2.0% (v/v).

[8] The method according to [6] or [7], wherein paraformaldehyde is used as a fixing agent in the step (2).

[9] The method according to any one of [1] to [8], wherein the surfactant is a nonionic surfactant.

[10] The method according to any one of [1] to [9], wherein the step (4) is carried out under a condition in which the concentration of formamide is from 15% (v/v) to 25% (v/v).

[11] The method according to any one of [1] to [10], wherein the labeled nucleic acid probe is a labeled peptide nucleic acid (PNA).

[12] The method according to any one of [1] to [11], wherein

following the step (4), performed is (4-1) a step of adding a second labeled antibody which has been labeled with a second labeling substance and is directed against the labeled part of the labeled nucleic acid probe and allowing the mixture to react, and

a cell labeled with both the first labeled antibody and the second labeling substance is detected in step (5).

[13] The method according to any one of [1] to [11], wherein

following the step (4), performed are (4-1) a step of adding a second labeled antibody which has been labeled with a second labeling substance and is directed against the labeled part of the labeled nucleic acid probe and allowing the mixture to react, and

(4-2) a step of adding a third labeled antibody which has been labeled with a third labeling substance and is directed against the second labeled antibody and allowing the mixture to react, and

a cell labeled with both the first labeled antibody and the third labeling substance is detected in step (5).

[14] The method according to [13], wherein

the second labeling substance is a fluorescent pigment selected from the group consisting of Alexa Fluor (registered trademark) 488, Oregon Green (registered trademark)-488, Rhodamine-123, Cy2, CYBR (registered trademark) Green I and EGFP, and

the third labeling substance is a fluorescent pigment selected from the group consisting of Alexa Fluor (registered trademark) 488, Oregon Green (registered trademark)-488, Rhodamine-123, Cy2, CYBR (registered trademark) Green I and EGFP.

[15] The method according to [13] or [14], wherein the second labeling substance and the third labeling substance are the same.

[16] A kit for detecting and identifying an Epstein-Barr virus-infected cell, comprising:

a first labeled antibody which has been labeled with a first labeling substance and is directed against a cell surface antigen specific to a target cell;

a labeled nucleic acid probe which is directed against a nucleic acid specific to Epstein-Barr virus;

a second labeled antibody which has been labeled with a second labeling substance and is directed against the labeled part of the labeled nucleic acid probe; and

a third labeled antibody which has been labeled with a third labeling substance and is directed against the second labeled antibody.

### BRIEF DESCRIPTION OF THE DRAWINGS

FIG. 1 shows the result of the experiment for amplifying a fluorescent intensity.

Using a fluorescently-labeled secondary antibody and a fluorescently-labeled tertiary antibody, amplification of a fluorescent intensity was attempted. Left: the result of the flow cytometry analysis for Raji, right: the result of the flow cytometry analysis for BJAB.

FIG. 2 shows the results of the flow cytometry analyses for EBV-positive B cell strains (Daudi and LCL), EBV-positive T cell strains (STN13 and SNT16: offered by Dr. Norio Shimizu) and EBV-positive NK cell strains (SNK6 and SNK10: offered by Dr. Norio Shimizu).

FIG. 3 shows the results of examination for the detection limit of FCM/ISH method.

FIG. 4 shows the results of triple staining of Raji, an EBV-positive B cell strain, by FCM/ISH method.

FIG. 5 shows the results of triple staining of SNK6, an EBV-positive NK cell strain, by FCM/ISH method.

FIG. 6 shows the results of double staining of human clinical samples (peripheral blood) by FCM/ISH method. Upper column: patient A, lower column: patient B.

FIG. 7 shows the results of detection by FCM/ISH method for human clinical samples (three examples of patients with chronic active EBV infection accompanying hydroa vacciniforme). The EBER-infected cell was observed in patient 1 (lower left), patient 2 (lower middle) and patient 3 (lower right) (1.7%, 4.8% and 25.9%, respectively). In the control (upper), the percentage of the positive cell was lower than 0.01%.

FIG. 8 shows identification of the EBV-infected cell by FCM/ISH method. The result of the flow cytometry analysis for the patient infected with chronic active

EBV accompanying hydroa vacciniforme (patient 1) is shown.

FIG. 9 shows identification of the EBV-infected cell by FCM/ISH method. The result of the flow cytometry analysis for the patient infected with chronic active EBV accompanying hydroa vacciniforme (patient 2) is shown.

FIG. 10 shows identification of the EBV-infected cell. The EBV-infected cell was identified by TCR gene reconstruction/magnetic beads method for human clinical samples (three examples of patients infected with chronic active EBV accompanying hydroa vacciniforme, an example of patient with B-lymphocyte hyperplasia after transplantation).

### BEST MODE FOR CARRYING OUT THE INVENTION

The first aspect of the present invention relates to a method for detecting and identifying a virus-infected cell (hereinafter sometimes referred to as “detection and identification method”). In the present specification, “detecting and identifying a virus-infected cell” means that a virus-infected cell is detected and identified at the same time. Furthermore, “identifying” as used herein means specifying the kind of the detected cell. Therefore, according to the detection and identification method of the present invention, a virus-infected cell can be detected, and information about the kind of the detected cell can be obtained.

The “virus” in the present invention is not specifically limited. Examples of the virus may include herpes viruses (herpes simplex virus-1 (HSV-1), herpes simplex virus-2 (HSV-2), varicella zoster virus (VZV), cytomegalovirus (CMV), human herpes virus-6 (HHV-6), human herpes virus-7 (HHV-7), Epstein-Barr virus virus (EBV), Kaposi's sarcoma-associated herpes virus (KSHV)), Retroviridae viruses (human immunodeficiency virus, Human T lymphotropic virus (HTLV) and the like) and parvovirus B19. Preferable “virus” is EBV. Namely, the present invention is preferably applied to the detection and identification of an EBV-infected cell.

In the detection and identification method of the present invention, the following steps (1) to (5) are carried out in this order:

(1) a step of adding a first labeled antibody which has been labeled with a first labeling substance and is directed against a cell surface antigen specific to a target cell to a sample and allowing the mixture to react;

(2) a step of fixing a protein in the presence of an RNA-stabilizing agent;

(3) a step of treating the sample with a surfactant;

(4) a step of adding a labeled nucleic acid probe for a nucleic acid specific to a target virus to cause hybridization; and

(5) a step of detecting a cell labeled with both the first labeled antibody and the labeled nucleic acid probe by flow cytometry.

### Step (1)

In the step (1), a predetermined antibody is prepared, and an antigen-antibody reaction is carried out to form an antigen-antibody complex. A labeled antibody is used which is directed against a cell surface antigen specific to a target cell. The “cell surface antigen specific to a target cell” refers to an antigen protein which is expressed on the cell surface of a target cell and is available as an indication for confirming the presence of the cell. Examples of the cell surface antigen may include CD2 (T cell, NK cell), CD3 (T cell), CD4 (helper T cell), CD8 (killer T cell), CD16 (NK cell), CD19 (B cell), CD20 (B cell), CD21 (B cell), CD34 (bone marrow stem cell), CD40 (B cell), CD40L (T cell), CD80 (B cell, dendritic cell, macrophage), HLA class II antigen (B cell, macrophage, T cell and the like), CD56 (NK cell), CD86 (B cell, dendritic cell, macrophage), CD161 (NK cell, T cell), TCRαβ (T cell), TCRγδ (T cell) and iNKT (NKT cell). Cell surface antigens are explained in detail, for example, in Zola H, Swart B, Banham A, et al. “CD molecules 2006—Human cell differentiation molecules.” Journal of Immunological Methods, 2006., Zola H, Swart B, Boumsell L, et al. “Human Leucocyte Differentiation Antigen nomenclature: update on CD nomenclature. Report of IUIS/WHO Subcommittee.” Journal of Immunological Methods, 275, 2004, p.p. 1-8., the official site (web page) of Human Cell Differentiation Molecules, and the like. For convenience of explanation, the “cell surface antigen specific to a target cell” is hereinafter abbreviated as “target antigen”. The “target cell” is suitably selected according to the kind of the target virus, application of the result of detection and identification and the like. Examples of the target cell may include B cells, T cells, NK cells, NKT cells, macrophages, dendritic cells, erythroblasts, bone marrow stem cells, myeloblasts, promyelocytes, myelocytes, metamyelocytes, polymorphonuclear leukocytes and megakaryoblasts.

The antibody which is directed against the target antigen can be prepared by utilizing an immunological technique, a phage display method, a ribosome display method or the like. The antibody which is directed against the target antigen may be polyclonal or monoclonal. Preparation of a polyclonal antibody by an immunological technique can be carried out according to the following procedures. A target antigen (or a part thereof) is prepared, and an animal such as a rabbit is immunized by using the antigen. An antigen prepared from a biological material (natural antigen) or a recombinant antigen may be used as the target antigen (or a part thereof). In order to enhance immune-eliciting effect, an antigen to which a carrier protein has been bound may be used. Examples of the carrier protein used may include KLH (Keyhole Limpet Hemocyanin), BSA (Bovine Serum Albumin), OVA (Ovalbumin) and the like. For binding of the carrier protein, carbodiimide method, glutalardehyde method, diazocondensation method, MBS (maleimidebenzoyloxysuccinic acid imide) method or the like can be used. Alternatively, also available is an antigen obtained by expressing CD46 (or a part thereof) as a fused protein with GST, β-galactosidase, maltose binding protein, histidine (His) tag or the like. Such fused protein can be conveniently purified by a versatile method.

If necessary, immunization is repeated, blood is collected at the time when an antibody titre has been sufficiently raised, and blood serum is obtained by a centrifugation treatment or the like. The obtained antiserum is subjected to affinity purification to give a polyclonal antibody.

On the other hand, a monoclonal antibody can be prepared by the following procedures. First, an operation of immunization is carried out according to similar procedures to those mentioned above. If necessary, immunization is repeated, and an antibody producing cell is isolated from the immunized animal at the time when an antibody titre has been raised sufficiently. The obtained antibody producing cell is then fused to a myeloma cell to give a hybridoma. The hybridoma is then monocloned, and a clone is selected which produces an antibody having high specificity against an objective protein. A culture liquid of the selected clone is purified to give an objective antibody. On the other hand, an objective antibody can be obtained by growing a hybridoma to a desired number or more, transplanting the hybridomas to the abdominal cavity of an animal (e.g., a mouse), growing the hybridomas in peritoneal fluid and purifying the peritoneal fluid. For the purification of the above-mentioned culture liquid or purification of the peritoneal fluid, affinity chromatography using Protein G, Protein A or the like is preferably used. Alternatively, also available is affinity chromatography in which an antigen is fixed on a solid phase. In addition, such methods may also be used as ion exchange chromatography, gel permeation chromatography, ammonium sulfate fractionation and centrifugation. These methods are used singly or in an optional combination.

The antibody used in the step (1) has been labeled. For convenience of explanation, the antibody is sometimes referred to as “first labeled antibody” in the following. Furthermore, the labeling substance used for labeling of the antibody is referred to as “first labeling substance” in the present specification. The kind of the first labeling substance is not specifically limited. In order to enable direct detection of the cell labeled with the first labeled antibody in the step (4) mentioned below, preferably used is a fluorescent pigment such as 7-AAD, Alexa Fluor (registered trademark) 488, Alexa Fluor (registered trademark) 350, Alexa Fluor (registered trademark) 546, Alexa Fluor (registered trademark) 555, Alexa Fluor (registered trademark) 568, Alexa Fluor (registered trademark) 594, Alexa Fluor (registered trademark) 633, Alexa Fluor (registered trademark) 647, Cy2, DsRED, EGFP, EYFP, FITC, PerCP™, R-Phycoerythrin, Propidium Iodide, AMCA, DAPI, ECFP, MethylCoumarin, Allophycocyanin, Cy™3, Cy™5, Rhodamine-123, Tetramethylrhodamine, Texas Red (registered trademark), PE, PE-Cy™5, PE-Cy™5.5, PE-Cy™7, APC, APC-Cy™7, Oregon Green, carboxyfluorescein, carboxyfluorescein diacetate and quantum dot. Alternatively, the cell surface antigen may be stained by two-steps of using a biotin-labeled antibody as the antibody used in the step (1) and reacting the antibody with fluorescence-labeled streptavidin. In the case when the cell labeled with the first labeled antibody is detected indirectly in the step (4), a labeling substance (e.g., biotin) other than fluorescent pigments can also be used. The “case when detected indirectly” as used herein means, for example, the case when the secondary antibody is detected by using an antibody which specifically recognizes the first labeling substance, an antibody which specifically recognizes the antibody part (e.g., Fc region) of the first labeled antibody (secondary antibody) or the like in combination, as well as the case when the tertiary antibody is detected by using an antibody which is directed against the secondary antibody (tertiary antibody), and the like. Using such secondary antibody and the like in combination can improve detection sensitivity.

Alternatively, when an antibody which is directed against the target antigen is commercially available, such antibody may also be utilized.

Two or more cell surface antigens may be targeted. In this case, two or more labeled antibodies which are different in cell surface antigen to be recognized are used. For example, if two or more cell surface antigens which are specific to certain kinds of cells are targeted, the detection in the step (5) is carried out by using expression of two or more cell surface antigens as indications, whereby a result of detection and identification with more higher reliability can be obtained. Alternatively, if targeting a cell surface antigen (one or two or more) which is specific to a certain kind of cell, and a cell surface antigen (one or two or more) which is specific to another kind of cell, results of detection and identification for the two kind of cells can be simultaneously obtained. For example, if targeting a cell surface antigen which is specific to a B cell and a cell surface antigen which is specific to a T cell, one can determine which the virus-infected cell is a B cell or a T cell, or not either, from the result of detection and identification in the step (5). It is also possible to determine three or more kinds of cell species in a similar manner by increasing the number of the cell surface antigen to be targeted.

Although the sample is not specifically limited, monocyte fractions of blood (e.g., peripheral blood, bone marrow aspirate), spinal fluid, pleural fluid and peritoneal fluid are preferably used as the sample. The method for the preparation of the sample may be carried out according to a general method.

The present invention can be widely applied for the purpose of investigation of possibility of morbidity of a specific viral disease, and the subject is not specifically limited. Examples of the subject may include a person who is suspected to be suffered from a specific viral disease, a person who has been diagnosed with a specific viral disease by other method, a patient who is suffered from a specific viral disease, a person who has received bone marrow transplantation, and a healthy person. As used herein, the “healthy person” refers to a person who is not diagnosed with a specific viral disease at the time when the detection and identification method of the present invention is applied.

The operations in the step (1), other reaction conditions and the like may follow general methods. For example, reference can be made to The Handbook of Staining and Bio-Imaging Experiments (Yodosha), The Handbook. A Guide to Fluorescent Probes and Labeling Technologies. 10^{th} ed. 2005 (Molecular Probes), and the like. The specific examples of operations and reaction conditions are shown in the Examples below.

### Step (2)

In the step (2) following the step (1), the protein is fixed in the presence of an RNA-stabilizing agent. In principle, the step (2) is carried out after the washing treatment.

The “RNA-stabilizing agent” is added for the purpose of preventing degragation of RNA which may accompany fixing of the protein. As the RNA-stabilizing agent, acetic acid is preferably used. Considering the effect of the protein on the fixing, the concentration of acetic acid is set. As a result of the examinatio by the inventors, it has been proved that a fine result is obtained when the concentration of acetic acid is from 0.5% (v/v) to 2.0% (v/v). Therefore, it is preferable to adopt this concentration range. Furthermore, the optimal concentration of acetic acid was 1% (v/v). Therefore, it is more preferable that the fixing is carried out under this concentration of acetic acid.

Although the reagent for fixing (fixing agent) is not specifically limited, it is preferable to use paraformaldehyde. The concentration of the fixing agent may be determined according to the fixing agent used. In the case when paraformaldehyde is adopted, the concentration may be from 3% (w/v) to 5% (w/v).

### Step (3)

In the step (3) which follows the step (2), the treatment is carried out with a surfactant. Namely, a membrane permeation treatment is carried out. In principle, the step (3) is carried out after the washing treatment.

So long as the desired purposes, i.e., that the permeabilities of the cell membrane and nuclear membrane are increased while the form of the cell is retained, and that intaking of the labeled nucleic acid probe becomes possible, are achievable, the kind and concentration of the surfactant are not specifically limited. A nonionic surfactant is suitable for a membrane permeation treatment for such purposes. Examples of the nonionic surfactant may include polyoxyethylene octyl phenyl ether, polyoxyethylene sorbitan monolaurate and polyoxyethylene lauryl ether, and specific examples may include TWEEN (registered trademark) 20, NP-40 (Nonidet P-40) and Triton (registered trademark) X-100. The concentration of the surfactant is, for example, from 0.1% (v/v) to 1.0% (v/v).

### Step (4)

In the step (4) which follows the step (3), the labeled nucleic acid probe for a nucleic acid specific to a target virus is added to cause hybridization. In principle, the step (4) is carried out after the washing treatment.

The sequence, kinds of component molecules and the like of the labeled nucleic acid probe are not specifically limited so long as it is specifically hybridized to a nucleic acid specific to a target virus. The “nucleic acid specific to a target virus” refers to a nucleic acid which is composed of a sequence which is inherent to the virus and is available for the detection of the virus. For example, when the target virus is EBV, EBV-encoded small RNA (EBER) falls within the “nucleic acid specific to a target virus”. EBER includes EBER-1 and EBER-2, and EBER-1 is preferable. This is because EBER-1 has about 10-fold higher amount of expression.

The sequences of EBER genes of various EBV virus strains are shown below (for the positions, statuses of registration in public databases and the like, see Tables 1 and 2 below).

EBER gene of Raji (EBER-1): SEQ ID NO: 1
EBER gene of B95-8 (EBER-1): SEQ ID NO: 2
EBER gene of GDI (EBER-1): SEQ ID NO: 3
EBER gene of AG876 (EBER-1): SEQ ID NO: 4
EBER gene of SNU-265 (EBER-1): SEQ ID NO: 5
EBER gene of SNU-20 (EBER-1): SEQ ID NO: 6
EBER gene of Akata (EBER-1): SEQ ID NO: 7

One example of the labeled nucleic acid probe (antisense probe) in the case when the “nucleic acid specific to a target virus” is EBER is shown below.

| | |
|---|---|
| GGCAGCGTAGGTCCT | (SEQ ID NO: 8) |

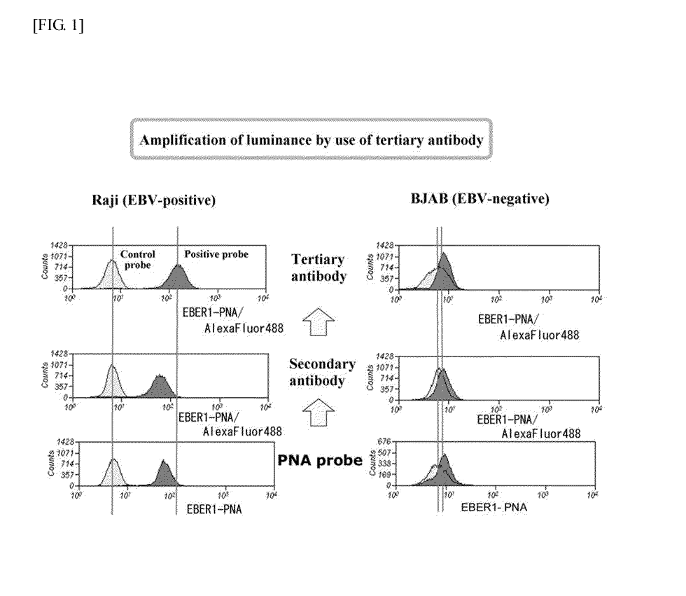

FIG. 1 shows the position of the probe sequence. Furthermore, as shown in Table 2, this probe sequence was also preserved in other EBV virus strains.

TABLE 1

| Cell | | Full length | Position | Position |
|---|---|---|---|---|
| line | Accession | (bp) | of EBER-1 | of probe |
| Raji | AJ507799 | 171823 | 6629~6795 | 6629~6643 |

TABLE 2

| | Accession | Full length (bp) | Position of EBER-1 | Position of probe |
|---|---|---|---|---|
| Complete genome | | | | |
| B95-8 | V01555 J 02700 | 172281 | 6629~6795 | 6629~6643 |
| | K01729 K01730 | | | |
| | V01554 X00498 | | | |
| | X00499 X00784 | | | |
| AG876 | DQ2779927 | 172764 | 6634~6800 | 6634~6648 |
| GD1 | AY961628 | 171657 | 6632~6798 | 6632~6646 |
| EBER-1 and | | | | |
| EBER-2 genes | | | | |
| SNU-265 | DQ883819 | 693 | 78~244 | 78~92 |
| SNU-20 | DQ883818 | 690 | 75~241 | 75~89 |
| EBER-1 gene | | | | |
| Akata | AB065135 | 167 | 1~167 | 1~15 |

It is preferable to use a labeled peptide nucleic acid (PNA) as the labeled nucleic acid probe in view of stability. This does not mean, however, that use of a labeled DNA probe, a labeled RNA probe and the like is interrupted. The labeled nucleic acid probe is designed so as to have a sequence which is complementary to a target sequence (i.e., a nucleic acid specific to a target virus). Therefore, hybridization with the target sequence under a suitable condition becomes possible. A higher complementarity of the sequence of the nucleic acid probe to the target sequence is generally preferable. The nucleic acid probe is designed so as to have a complementarity of, preferably 90% or more, more preferably 95% or more, further preferably 99% or more, and most preferably 100%.

In addition, a labeled PNA probe targeting for EBER (Epstein-Barr Virus (EBER) PNA Probe/Fluorescein, Code No. Y5200, Dako) is commercially available. When the target virus is EBV, the probe can be used as the “nucleic acid-labeled probe”.

As used herein, the “peptide nucleic acid (PNA)” is a compound having a structure in which a nucleic acid base is bound to a polypeptide backbone. Examples of the polypeptide backbone may include those having 2-aminoethylglycine as a backbone unit, but the PNA in the present invention is not limited thereto. PNA shows resistance against a nuclease, and has higher stability than those of DNA and RNA. Furthermore, it generally shows high resistance against a peptidase. PNA can be hybridized to DNA or RNA. In general, a PNA-DNA or PNA-RNA complex have higher stability than those of a DNA-DNA complex and a DNA-RNA complex. Therefore, in the case of the present invention in which various treatments are carried out until the detection, a PNA probe is preferable.

The labeling substance used for labeling of the nucleic acid probe is not specifically limited. If the labeled nucleic acid probe is directly detected by flow cytometry in the next step (5) (i.e., in the case when the labeling substance used for the labeled nucleic acid probe is detected by flow cytometry, whereby a cell labeled with the labeled nucleic acid probe is detected), a fluorescent pigment is selected as the labeling substance. Examples of the fluorescent pigment may include 7-AAD, Alexa Fluor (registered trademark) 488, Alexa Fluor (registered trademark) 350, Alexa Fluor (registered trademark) 546, Alexa Fluor (registered trademark) 555, Alexa Fluor (registered trademark) 568, Alexa Fluor (registered trademark) 594, Alexa Fluor (registered trademark) 633, Alexa Fluor (registered trademark) 647, Cy™2, DsRED, EGFP, EYFP, FITC, PerCP™, R-Phycoerythrin, Propidium Iodide, AMCA, DAPI, ECFP, MethylCoumarin, Allophycocyanin, Cy™3, Cy™5, Rhodamine-123, Tetramethylrhodamine, Texas Red (registered trademark), PE, PE-Cy™5, PE-Cy™5.5, PE-Cy™7, APC, APC-Cy™7, Oregon Green, carboxyfluorescein, carboxyfluorescein diacetate and quantum dot.

A labeling substance other than the fluorescent pigment (e.g., biotin) may be used in the case when the labeled nucleic acid probe is not directly detected by flow cytometry in the next step (5) (e.g., in the case of the embodiment in which the following step (4-1) is carried out).

As shown in the following Examples, as a result of the examination by the present inventors, it was proved that a fine result was brought about when the hybridization reaction was carried out under the condition of the concentration of formamide from 15% (v/v) to 25% (v/v). Therefore, the hybridization reaction is preferably carried out under the condition of the concentration of formamide in this concentration range. Furthermore, the optimal concentration of formamide was 20% (v/v). Therefore, more preferably, the hybridization reaction is carried out by this concentration of formamide. When the concentration of formamide is too high, the antigen-antibody composite formed in the step (1) drops off and is modified, and when the concentration of formamide is too low, the specificity of hybridization is deteriorated.

The operations in the steps (2) to (4), other reaction conditions and the like may follow conventional methods. For example, reference can be made to T. Just et al., J Virol Methods 73 (1998) 163-174, The Handbook. A Guide to Fluorescent Probes and Labeling Technologies. 10^{th} ed. 2005 (Molecular Probes) and the like. The specific examples of the operations, reaction conditions and the like are shown in the following Examples.

### Step (5)

In the step (5) which follows the step (4), a cell labeled with both the first labeled antibody and the labeled nucleic acid probe is detected by flow cytometry (FCM). In principle, the step (5) is carried out after the washing treatment.

If as a result of the step (5), a cell labeled with both the first labeled antibody and the labeled nucleic acid probe is detected, it is considered that a virus-infected cell is present in the sample, and that the species of the cell is same as that of the target cell. On the other hand, if a cell labeled with the labeled nucleic acid probe is detected but a cell labeled with the first labeled antibody is not detected, it is considered that a virus-infected cell is present in the sample but the species of the cell differs from that of the target cell. In addition, a detection result that a cell labeled with the labeled nucleic acid probe is not detected (irrespective of the detection result of the cell labeled with the first labeled antibody) shows that a virus-infected cell is absent in the sample.

As mentioned above, if two or more cell surface antigens are targeted in the step (1), the species of the cells can be distinguished using expression of two or more cell surface antigens as indication.

The apparatuses for the flow cytometry analysis are sold by, for example, BeckmanCoulter, Becton, Dickinson and Company Japan and the like, and these can be utilized for the present invention. The basic operation methods, analysis conditions and the like may follow the written instructions attached to the apparatus. Furthermore, there are many articles and books relating to flow cytometry analyses, and for example, Cao T M, et al. Cancer. 2001 Jun. 15; 91 (12): 2205-13., Storek K J, et al. Blood 97: 3380-3389, WEIR'S HANDBOOK OF EXPERIMENTAL IMMUNOLOGY Vol. II <Blackwell Science>, Little MT and R. Storb Nture Reviews Cancer 2002 2: 231-238 and the like are used as references.

When a fluorescent labeled antibody is used as the first labeled antibody, determination of presence or absence and/or quantification of a cell labeled with the first labeled antibody can be achieved by directly detecting the fluorescence emitted by the fluorescent labeled antibody. Similarly, in the case when the fluorescent labeled nucleic acid probe is used as the labeled nucleic acid probe, determination of presence or absence and/or quantification of a cell labeled with the labeled nucleic acid probe can be achieved by directly detecting the fluorescence emitted by the fluorescent labeled nucleic acid probe. Instead of directly detecting the labeled nucleic acid probe in such a manner, indirect detection may be carried out as in the embodiment shown below.

In one embodiment of the present invention, after the step (4) (prior to the step (5)), performed is a step of adding an antibody which has been labeled with a second labeling substance and is directed against the labeled part of the labeled nucleic acid probe (second labeled antibody) and allowing the mixture to react (step (4-1)). Furthermore, in the following step (5), the label which is used for the second labeled antibody is utilized for detecting a cell labeled with a labeled nucleic acid probe. Therefore, in this embodiment, the first labeling substance (for the detection of a cell labeled with the first labeled antibody) and the second labeling substance (for the detection of a cell labeled with the target nucleic acid probe) are objects of the detection.

When indirect detection is carried out by using the second labeled antibody in this manner, the signal is enhanced, and the detection sensitivity and S/N ratio are improved.

The second labeled antibody may be polyclonal or monoclonal. As the second labeling substance, available is 7-AAD, Alexa Fluor (registered trademark) 488, Alexa Fluor (registered trademark) 350, Alexa Fluor (registered trademark) 546, Alexa Fluor (registered trademark) 555, Alexa Fluor (registered trademark) 568, Alexa Fluor (registered trademark) 594, Alexa Fluor (registered trademark) 633, Alexa Fluor (registered trademark) 647, Cy™2, DsRED, EGFP, EYFP, FITC, PerCP™, R-Phycoerythrin, Propidium Iodide, AMCA, DAPI, ECFP, MethylCoumarin, Allophycocyanin, Cy™3, Cy™5, Rhodamine-123, Tetramethylrhodamine, Texas Red (registered trademark), PE, PE-Cy™5, PE-Cy™5.5, PE-Cy™7, APC, APC-Cy™7, Oregon Green, carboxyfluorescein, carboxyfluorescein diacetate, or quantum dot. As the second labeling substance, preferably used is a fluorescent pigment selected from the group consisting of Alexa Fluor (registered trademark) 488, Oregon Green (registered trademark)-488, Rhodamine-123, Cy2, CYBR (registered trademark) Green I and EGFP.

In another embodiment of the present invention, after the step (4) (prior to the step (5)), performed are a step of adding an antibody which has been labeled with a second labeling substance and is directed against the labeled part of the labeled nucleic acid probe (second labeled antibody) and allowing the mixture to react (step (4-1)), and a step of adding an antibody which has been labeled with the third labeling substance and is directed against the second labeled antibody (third labeled antibody) and allowing the mixture to react (step (4-2)). Then, in the following step (5), the label which is used for the third labeled antibody is utilized for detecting a cell labeled with the labeled nucleic acid probe. Therefore, in this embodiment, the first labeling substance (for the detection of a cell labeled with the first labeled antibody) and the third labeling substance (for the detection of a cell labeled with the target nucleic acid probe) are objects of the detection. According to this embodiment, the signal is enhanced stepwise, and the detection sensitivity and S/N ratio are further improved.

The third labeled antibody specifically recognizes the second labeled antibody. For example, when a rabbit antibody is used as the second labeled antibody, an anti-rabbit antibody may be used as the third labeled antibody. Similarly to the second labeled antibody, the third labeled antibody may be polyclonal or monoclonal. As the third labeling substance, available is 7-AAD, Alexa Fluor (registered trademark) 488, Alexa Fluor (registered trademark) 350, Alexa Fluor (registered trademark) 546, Alexa Fluor (registered trademark) 555, Alexa Fluor (registered trademark) 568, Alexa Fluor (registered trademark) 594, Alexa Fluor (registered trademark) 633, Alexa Fluor (registered trademark) 647, Cy™2, DsRED, EGFP, EYFP, FITC, PerCP™, R-Phycoerythrin, Propidium Iodide, AMCA, DAPI, ECFP, MethylCoumarin, Allophycocyanin, Cy™3, Cy™5, Rhodamine-123, Tetramethylrhodamine, Texas Red (registered trademark), PE, PE-Cy™5, PE-Cy™5.5, PE-Cy™7, APC, APC-Cy™7, Oregon Green, carboxyfluorescein, carboxyfluorescein diacetate, quantum dot or the like. As the third labeling substance, preferably used is a fluorescent pigment selected from the group consisting of Alexa Fluor (registered trademark) 488, Oregon Green (registered trademark)-488, Rhodamine-123, Cy2, CYBR (registered trademark) Green I and EGFP.

Here, it is preferable that the second labeling substance and the third labeling substance are the same. Namely, used are the second labeled antibody and the third labeled antibody which are labeled with the same labeling substance. By doing so, the signal is further enhanced.

According to the detection and identification method of the present invention, a virus-infected cell can be detected and information about the kind of the infected cell can be obtained. The result of the detection and identification can be used for the diagnosis of, prognosis of contracting, effect of treatment of, viral diseases, and the like. For example, if, when the present invention is applied to the detection and identification of an EBV virus-infected cell, it is proved that a B cell is present as a virus-infected cell in a sample, one can determine that a subject is affected or highly possible to be affected by opportunistic lymphoma, Hodgkin's lymphoma or the like. On the other hand, a result of the detection and identification that a T cell is present as a virus-infected cell in a sample enables the diagnosis of, and expectation of contracting T cell lymphoma and T cell leukemia. Similarly, a result of the detection and identification that an NK cell is present as a virus-infected cell in a sample enables the diagnosis of, and expectation of contracting nasal NK lymphoma and NK leukemia.

Specifically, the detection and identification method of the present invention is highly useful in that it can be utilized for early diagnosis of viral diseases. If early diagnosis becomes possible, medical intervention in an early stage becomes possible, which leads to improvement of treatment effect, improvement of prognosis and the like.

The second aspect of the present invention relates to a kit which is utilized for the detection and identification method of the present invention. The kit of the present invention comprises the first labeled antibody and the labeled nucleic acid probe as essential constitutional elements. One embodiment comprises the second labeled antibody and/or the third labeled antibody. Reagents (a buffer fluid, a washing fluid, a fixing agent, an RNA-stabilizing agent, a surfactant, a solution for hybridization and the like) and/or apparatuses or instruments (a container, a reaction apparatus and the like) which are required for the respective operations and reactions (antigen-antibody reaction, fixing, membrane permeation treatment, hybridization reaction and the like) may also be incorporated in the kit. In general, an instruction manual is attached to the kit of the present invention.

### EXAMPLES

Aiming at establishing a method for detecting and identifying an EBV-infected cell in a suspension cell system, the following studies were done.

### 1. Determination of Fixing Condition (Examination for Concentration of Acetic Acid)

A fixing condition for stabilizing both a protein and RNA is important for continuously carrying out an antigen-antibody reaction to a protein, a surface antigen, and a hybridization reaction to RNA, EBER-1.

Many fixing solutions for protein (formalin, paraformaldehyde, commercially available fix solutions and the like) were tried, and examination was made on temperature conditions, reaction times and the like. As a result, it was finally found that the case when fixing was carried out at 4° C. for 40 minutes by using 1% (v/v) acetic acid/4% (w/v) paraformaldehyde/PBS was optimal. The results as the bases are shown in below.

Raji cell, an EBV-infected cell strain, was reacted with an anti-CD21 antibody labeled with PE (Raji cell is surface antigen CD21-positive since it is a B cell), and the cell was fixed under various conditions (paraformaldehyde was 4% (w/v), and the concentration of acetic acid was varied with 1% increments), and in situ hybridization was then carried out by using a FITC-labeled EBER-1-PNA probe (Dako, Y5200). Then, fluorescent intensity was measured by flow cytometry (FACS Caliber manufactured by Becton, Dickinson and Company was used). The fluorescent intensity of PE was highest when the concentration of acetic acid was 1% (Table 3). In accordance with the increase of the concentration of acetic acid, the fluorescent intensity of FITC was increased, but sufficient fluorescent intensity was obtained even when the concentration of acetic acid was 1% (Table 3). Although not shown here, as a result of similar examination using concentrations of acetic acid varied with 0.5% increments, both the protein and RNA could be detected finely when the concentration of acetic acid was in the range of from 0.5% (v/v) to 2% (v/v).

TABLE 3

Fluorescent intensity

| | PE-labeled | FICT-labeled |
|---|---|---|
| | anti-CD21 | EBER1-PNA |
| Acetic acid 0% | 166.14 | 29.42 |
| Acetic acid 1% | 194.10 ↑ | 61.93 |
| Acetic acid 2% | 162.82 | 93.36 |
| Acetic acid 3% | 119.53 | 113.56 |
| Acetic acid 4% | 73.65 | 162.79 |

### 2. Determination of Hybridization Condition (Examination for Concentration of Formamide)

In order to establish a detection and identification method comprising a combination of flow cytometry and in situ hybridization (FCM/ISH method), it is also important to determine a condition under which the PNA probe is specifically hybridized to EBER-1 and the surface antigen-antibody composite does not drop off and is not modified. As a result of intensive examination, it was found that the optimal condition for the hybridization reaction was a condition for carrying out the reaction at 56° C. for 60 minutes in the presence of 10 mM of NaCl, 5 mM of Na₂EDTA, 50 mM of Tris-HCl (pH 7.5) and 20% (v/v) of formamide. The result which serves as a basis is shown below.

Raji cell, an EBV-infected cell strain, was reacted with an anti-CD21 antibody labeled with PE, and the cell was fixed under various conditions. Thereafter in situ hybridization was carried out, and fluorescence was measured by flow cytometry. The concentration of formamide which had the most effect on the decrease of a background was examined from 0% (v/v) to 30% (v/v) with 5% increments. As shown in below, it was apparent that detection of the PE-labeled anti-CD21 antibody was significantly deteriorated and the surface antigen-antibody composite dropped off and was modified when formamide was 25% (v/v) or more (Table 4). Meanwhile, both the surface antigen and EBER-1 could be finely detected when the concentration of formamide was in the range of from 15% (v/v) to 25% (v/v), and they had the most favorable balance at 20% (v/v).

TABLE 4

Fluorescent intensity

| | PE-labeled | FITC-labeled |
|---|---|---|
| | anti-CD21 | EBER1-PNA* |
| Formamide 15% | 98.04 | 15.07 |
| Formamide 20% | 62.83 | 17.37 |
| Formamide 25% | 39.80 ↓ | 16.51 |
| Formamide 30% | 20.87 ↓ | 23.39 |

*Since fluorescence was not amplified, the fluorescent intensity of FITC was low.

### 3. Improvement of Detection Sensitivity by Amplification of Fluorescent Intensity

Since a commercially available PNA probe (FITC labeled EBER-1-PNA probe (Dako, Y5200)) uses FITC which emits weak fluorescence, the signal thereof is weak and difficult to detect even by flow cytometry.

In order to enhance the signal of the PNA probe labeled with FITC, a secondary antibody, Alexa Fluor (registered trademark) 488-labeled Anti-FITC rabbit IgG (Invitrogen: A11090) and a tertiary antibody, Alexa Fluor (registered trademark) 488-labeled Anti-rabbit goat IgG (Invitrogen: A11034) were each reacted at room temperature for 20 minutes after the hybridization reaction. As a result, it was found that the signal/background ratio was high and a significantly high signal was obtained when the concentrations of the secondary antibody and the tertiary antibody were both 2.5 μg/mL. A part of the results which serve as bases is shown below.

An EBV-positive Raji cell and an EBV-negative BJAB cell were fixed and subjected to in situ hybridization using an FITC-labeled EBER-1-PNA probe (Dako, Y5200), and the fluorescent intensity when amplified with only the secondary antibody or with both the secondary antibody and the tertiary antibody was compared with the fluorescent intensity prior to the amplification. In Raji cell, significant amplification of the signal was recognized when the tertiary antibody was used (FIG. 1, left). On the other hand, amplification of the fluorescent intensity was not observed in BJAB cell (FIG. 1, right).

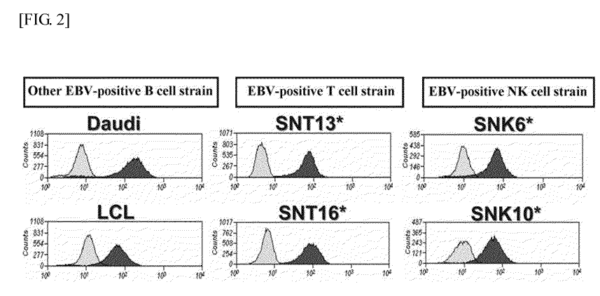

Furthermore, it was shown that an EBV-infected cell could be detected with high sensitivity also in other EBV-positive B cell strains (Daudi and LCL), EBV-positive T cell strains (STN13 and SNT16: offered by Dr. Norio Shimizu) and EBV-positive NK cell strains (SNK6 and SNK10: offered by Dr. Norio Shimizu) by enhancing the signal of the PNA probe (FIG. 2).

### 4. Examination for Detection Limit of EBV-positive Cell

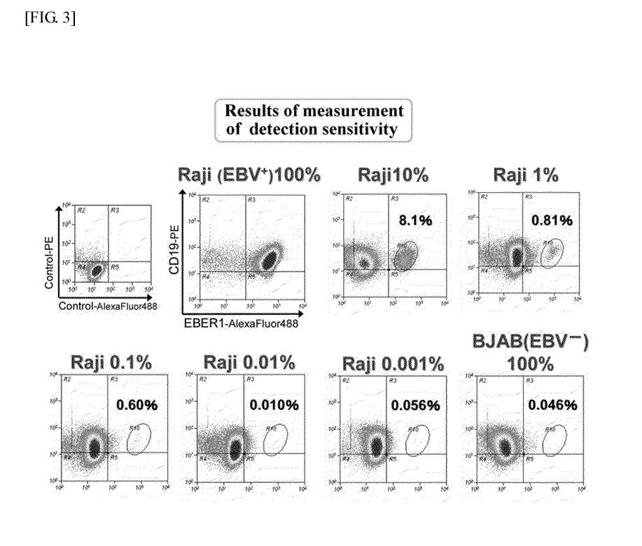

FCM/ISH method was carried out by using samples in which Raji, an EBV-positive B cell strain, and BJAB, an EBV-negative B cell strain, were mixed by various ratios (Raji 100%, 10%, 1%, 0.1%, 0.01% and 0.001%) to examine the detection limit of the EBV-positive cell. As shown in FIG. 3, the mixing ratio of the EBV-positive cell could be detected up to 0.01% (in 0.001%, no difference from 0% [BJAB 100%] was observed). This shows that even one EBV-infected cell in 10,000 peripheral blood mononuclear cells can be detected by this system.

### 5. Multiple Staining by FCM/ISH Method

The optimal condition found by the above-mentioned examination was adopted to each reaction, and multiple staining was tried in a cell surface antigen and EBER-1 which is a virus specific mRNA by FCM/ISH method using an EBV-positive B cell strain, a T cell strain, an NK cell strain and a clinical material (peripheral blood mononuclear cell) as samples. The surface antigen was stained by using two kinds of fluorescent pigments PE and PC5. In labeling of EBER-1, a secondary antibody, Alexa Fluor (registered trademark) 488-labeled Anti-FITC rabbit IgG (Invitrogen: A11090), then a tertiary antibody, Alexa Fluor (registered trademark) 488-labeled Anti-rabbit goat IgG (Invitrogen: A11034) were reacted after the hybridization reaction using the FITC labeled EBER-1-PNA probe. Thus, multiple staining with PE, PC5 and Alexa Fluor (registered trademark) 488 was achieved. The specific operation procedures are shown below.

### (1) Adjustment of Number of Cells

The number of cells was adjusted with PBS/2% FCS so as to became 1×10⁶ cells per 1 ml. 200 μl each was transferred to a 1.5 ml tube (2×10⁵ cells per one tube). After a treatment by centrifugation (5000 rpm, 1 minute), the supernatant was removed by aspiration. The peripheral blood of a patient used as a clinical material was collected after obtaining an agreement with the patient and a person with parental authority, and monocytes were separated according to a conventional method and used for the experiment. In addition, the following operations were carried out in a darkened room.

### (2) Antigen-antibody Reaction

The cells were resuspended in 40 μl of PBS/2% FCS, 10 μl of a fluorescent-labeled (PE or PC5) antibody was added, and the reaction was carried out at 4° C. for 60 minutes. 1 ml of PBS/2% FCS was added, and the mixture was centrifuged (5000 rpm, 1 minute) to discard the supernatant. The washing operation was carried out twice in total.

### (3) Fixing

300 μl of 1% (v/v) acetic acid/4% (w/v) paraformaldehyde/PBS was added, pipetted lightly, and reacted at 4° C. for 40 minutes.

### (4) Washing of Cells

After centrifugation (6000 rpm, 2 minutes), the supernatant was discarded. 1 ml of PBS was added, and the mixture was stirred and centrifuged (6000 rpm, 2 minutes) to remove the supernatant by suction.

### (5) Membrane Permeation Treatment

50 μl of 0.5% (v/v) TWEEN (registered trademark) 20/PBS was charged, and left at room temperature for 10 minutes.

### (6) Hybridization

After centrifugation (5000 rpm, 1 minute), the supernatant was removed by suction. 12.5 μl of a buffer (final concentrations were 10 mM of NaCl, 5 mM of Na₂EDTA, 50 mM of Tris-HCl (pH 7.5), 20% (v/v) of formamide) and 25 μl of a probe (Epstein-Barr Virus (EBER) PNA Probe/Fluorescein (Code No. Y5200, Dako) or a negative control PNA probe attached to the probe) were added, and the cells were resuspended and reacted at 56° C. for 60 minutes.

### (7) Washing of Cells

1 ml of 0.5% (v/v) TWEEN (registered trademark) 20/PBS was added, and the mixture was stirred and then reacted at 56° C. for 10 minutes. After centrifugation (5000 rpm, 1 minutes), the supernatant was discarded. 1 ml of 0.5% (v/v) TWEEN (registered trademark) 20/PBS was added, and the mixture was stirred and then reacted at 56° C. for 30 minutes.

### (8) Reaction of Alexa-labeled Secondary Antibody

200 μl of Alexa Fluor (registered trademark) 488-labeled Anti-FITC rabbit IgG (Invitrogen: A11090) was added. The reaction was carried out at room temperature for 20 minutes (the final concentration of the antibody was 2.5 μg/ml). 1 ml of 0.5% (v/v) TWEEN (registered trademark) 20/PBS was added, and the mixture was stirred and then centrifuged (5000 rpm, 1 minutes) to discard the supernatant. The washing operation was carried out twice in total.

### (9) Reaction of Alexa-labeled Tertiary Antibody

200 μl of Alexa Fluor (registered trademark) 488-labeled Anti-rabbit goat IgG (Invitrogen: A11034) was added. Reaction was carried out at room temperature for 20 minutes (the final concentration of the antibody was 2.5 μg/ml). 1 ml of 0.5% (v/v) TWEEN (registered trademark) 20/PBS was added, and the mixture was stirred and centrifuged (5000 rpm, 1 minutes) to discard the supernatant. This washing operation was carried out twice in total.

### (10) Flow Cytometry Analysis

0.5 ml of 0.5% (v/v) TWEEN (registered trademark) 20/PBS was added, and the mixture was stirred and then subjected to flow cytometry analysis (FACS Caliber manufactured by Becton, Dickinson and Company was used).

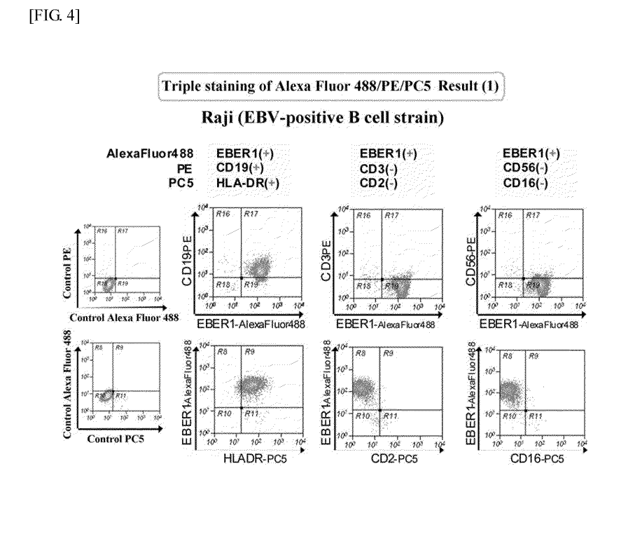

FIG. 4 shows the results of the detection for Raji. For Raji which is an EBV-positive B cell strain, CD19 and HLA-DR of the surface antigen were positive, and CD2, CD3, CD16 and CD56 were negative.

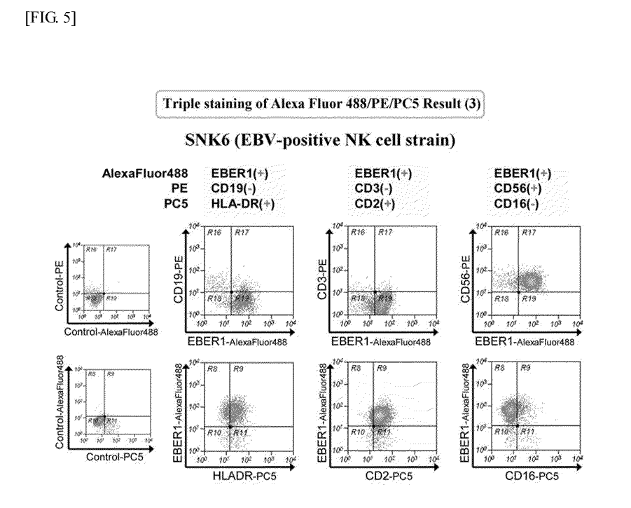

FIG. 5 shows the result of the detection for SNK6. For SNK6 which is an EBV-positive NK cell strain, CD2, CD56 and HLA-DR of the surface antigen were positive, and CD3, CD16 and CD19 were negative.

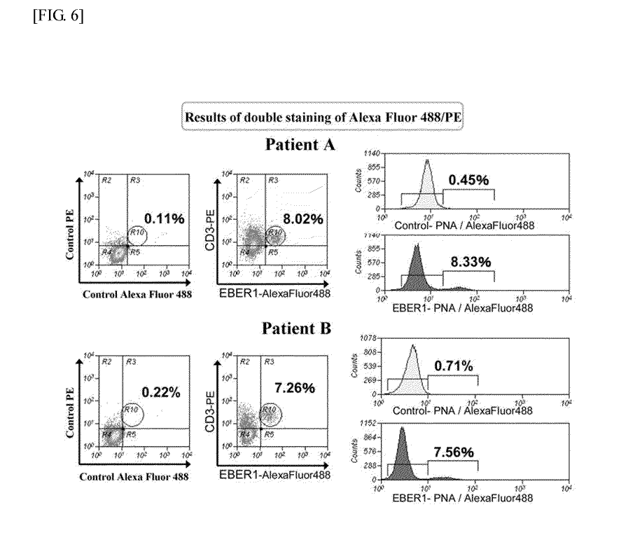

FIG. 6 shows the results of the detection for human clinical samples. Multiple staining of EBER-1 and the cell surface antigen was also possible for a human clinical samples (peripheral blood of chronic active EBV-infected patients), and the EBV-infected cell could be identified. About 8% and 7%, respectively, of the peripheral blood of the patients A and B were infected by EBV, and all of the infected cells were considered to be CD3-positive T cells.

Using human clinical samples (three examples of chronic active EBV-infected patients accompanying hydroa vacciniforme, an example of a patient with B-lymphocyte hyperplasia after transplantation, and five examples of healthy persons who had been already infected with EBV), an attempt was made to detect and identify an EBER-positive cell in human peripheral monocyte. Chronic active EBV infection accompanying hydroa vacciniforme is an EBV-related lymphocyte proliferative disease accompanying solar hypersensitivity, and is infrequently observed among children in Japan and Latin America. The disease is characterized by emergence of papulae and bullae which turn into ulcers and scars. The disease sometimes accompanies systemic symptoms such as fever, swelling of lymph nodes and hepatosplenomegaly. Furthermore, EBER positive lymphocytes (although there are various theories, they are mainly T cells) are collected under the skin.

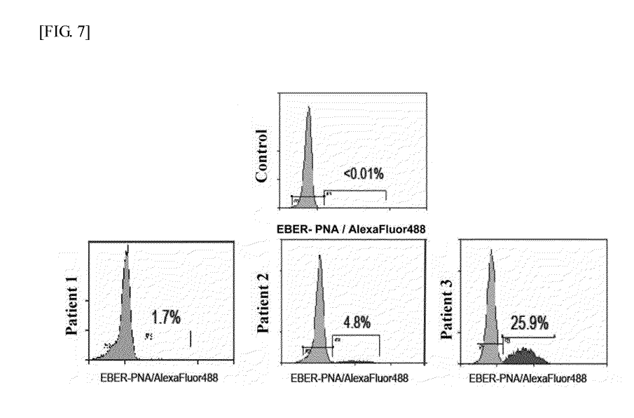

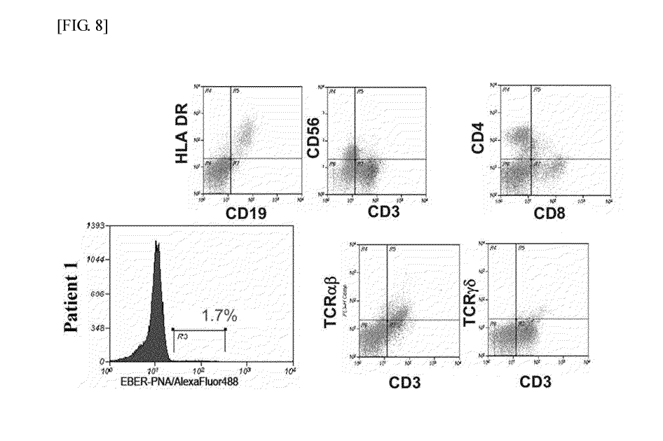

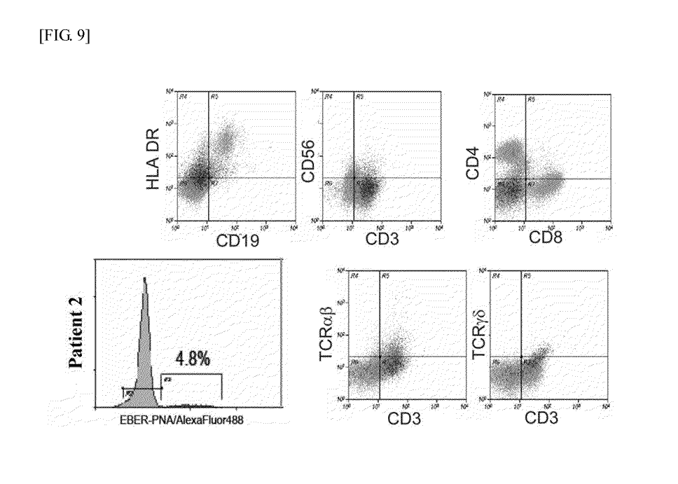

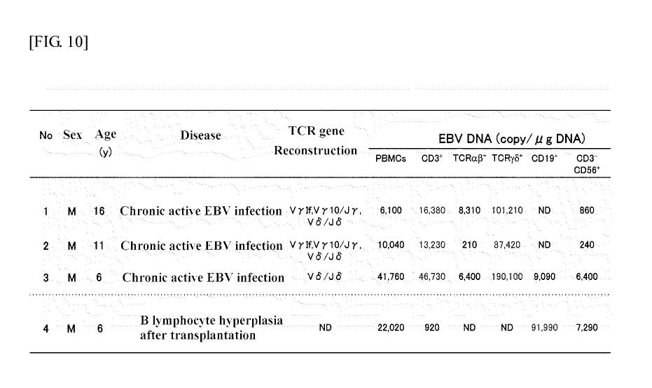

As shown in FIG. 7, 1.7 to 25.9% of EBER positive cell was observed in the peripheral blood of the patients infected with chronic active EBV accompanying hydroa vacciniforme. Furthermore, in these patients, CD3⁺CD4⁻CD8⁻TCRγδ⁺ T cell was infected with EBV (FIGS. 8 to 10). Accordingly, it has been shown that the method of the present invention is useful for not only diagnoses of EBV-related diseases but also for clarification of pathology of onset.

### INDUSTRIAL APPLICABILITY

According to the detection and identification method of the present invention, a virus-infected cell can be detected and identified specifically with high sensitivity in suspension cell systems. Namely, not only detection of a virus-infected cell in a sample but also identification of the kind of the infected cell is possible. On the other hand, the period required for the series of processes is short in the detection and identification method of the present invention, and thus the present invention is superior to conventional methods in terms of swiftness. Furthermore, the present invention is highly versatile since it can essentially be carried out if one has an instrument for flow cytometry.

The present invention is specifically useful for detection and identification of an EBV-infected cell. Opportunistic lymphoma is a lethal EBV-related disease which accompanies AIDS and transplantation of organs or bone marrow. The bases for the diagnosis of opportunistic lymphoma are increase of EBV-infected cells in peripheral blood, and that the infected cell is a B cell. Previously, since an EBV-infected cell in a tissue was identified on biopsy of a lymph node, the identification was highly invasive and the diagnosis required a longer time period. If the present invention is applied by using peripheral blood as a sample, an infected cell can be quantified and identified at the same time in a quite short time period without invasion. In the meantime, in recent, Rituximab which is a B cell monoclonal antibody is used for the treatment of opportunistic lymphoma. If the result of the detection and identification method of the present invention is utilized, early diagnosis of opportunistic lymphoma becomes possible, and the treatment can be initiated at an earlier stage. Furthermore, the detection and identification method of the present invention is also useful for the determination of the treatment effect. The EBV-related diseases for which application of the detection and identification method of the present invention is conceived cover a wide variety of diseases including nasal NK lymphoma, Hodgkin's lymphoma, NK leukemia, T cell lymphoma, chronic active EBV infection, infectious mononucleosis and the like, besides opportunistic lymphoma.

By suitably choosing/changing the nucleic acid probe to be used, the present invention can be applied to various viral diseases (viral diseases caused by infection of a blood cell such as HIV infection and cytomegarovirus). Accordingly, the present invention has quite high versatility and applicability, and is expected of substantial contribution to the fields of the diagnosis and treatment of virus-related diseases.

The present invention is not limited at all by the explanations of the embodiments and Examples of the above-mentioned invention. Various modified embodiments are also encompassed in the present invention to the extent that a person skilled in the art can readily conceive, without departing from the description of the claims.

All contents of the articles, patent publications and granted patent publications disclosed in the present specification are incorporated herein by reference.

1. A method of detecting and identifying a virus-infected cell, comprising the following steps (1) to (5):
(1) a step of adding a first labeled antibody which has been labeled with a first labeling substance and is directed against a cell surface antigen specific to a target cell to a sample and allowing the mixture to react;
(2) a step of fixing a protein in the presence of an RNA-stabilizing agent;
(3) a step of treating the sample with a surfactant;
(4) a step of adding a labeled nucleic acid probe for a nucleic acid specific to a target virus to cause hybridization; and
(5) a step of detecting a cell labeled with both the first labeled antibody and the labeled nucleic acid probe by flow cytometry.

2. The method according to claim 1, wherein the target virus is Epstein-Barr virus.

3. The method according to claim 2, wherein the nucleic acid specific to the target virus is a small RNA encoded by Epstein-Barr virus (EBER).

4. The method according to claim 1, wherein the target cell is B cell, T cell or NK cell.

5. The method according to claim 4, wherein the sample is a blood sample.

6. The method according to claim 1, wherein the RNA-stabilizing agent is acetic acid.

7. The method according to claim 6, wherein the step (2) is carried out under a condition in which the concentration of acetic acid is from 0.5% (v/v) to 2.0% (v/v).

8. The method according to claim 6, wherein paraformaldehyde is used as a fixing agent in the step (2).

9. The method according to claim 1, wherein the surfactant is a nonionic surfactant.

10. The method according to claim 1, wherein the step (4) is carried out under a condition in which the concentration of formamide is from 15% (v/v) to 25% (v/v).

11. The method according to claim 1, wherein the labeled nucleic acid probe is a labeled peptide nucleic acid (PNA).

12. The method according to claim 1, wherein
(4-1) a step of adding a second labeled antibody which has been labeled with a second labeling substance and is directed against the labeled part of the labeled nucleic acid probe and allowing the mixture to react is carried out following the step (4), and
a cell labeled with both the first labeled antibody and the second labeling substance is detected in step (5).

13. The method according to claim 1, wherein
(4-1) a step of adding a second labeled antibody which has been labeled with a second labeling substance and is directed against the labeled part of the labeled nucleic acid probe and allowing the mixture to react, and
(4-2) a step of adding a third labeled antibody which has been labeled with a third labeling substance and is directed against the second labeled antibody and allowing the mixture to react, are carried out following the step (4), and
a cell labeled with both the first labeled antibody and the third labeling substance is detected in step (5).

14. The method according to claim 13, wherein
the second labeling substance is a fluorescent pigment selected from the group consisting of Alexa Fluor (registered trademark) 488, Oregon Green (registered trademark)-488, Rhodamine-123, Cy2, CYBR (registered trademark) Green I and EGFP, and
the third labeling substance is a fluorescent pigment selected from the group consisting of Alexa Fluor (registered trademark) 488, Oregon Green (registered trademark)-488, Rhodamine-123, Cy2, CYBR (registered trademark) Green I and EGFP.

15. The method according to claim 13, wherein the second labeling substance and the third labeling substance are the same.

16. A kit for detecting and identifying an Epstein-Barr virus-infected cell, comprising:
a first labeled antibody which has been labeled with a first labeling substance and is directed against a cell surface antigen specific to a target cell;
a labeled nucleic acid probe which is directed against a nucleic acid specific to Epstein-Barr virus;
a second labeled antibody which has been labeled with a second labeling substance and is directed against the labeled part of the labeled nucleic acid probe; and
a third labeled antibody which has been labeled with a third labeling substance and is directed against the second labeled antibody.
